(19) United States
(12) Patent Application Publication
(10) Pub. No.: US 2001/0038834 A1
(43) Pub. Date: Nov. 8, 2001

(54) BONE CANCER THERAPY

(76) Inventors: GARY BALIAN, CHARLOTTESVILLE, VA (US); QUANJUN CUI, CHARLOTTESVILLE, VA (US); LELAND W.K. CHUNG, LOVINGSTON, VA (US); CHINGHAI KAO, CHARLOTTESVILLE, VA (US); THOMAS A. GARDNER, CHARLOTTESVILLE, VA (US)

Correspondence Address: MCGUIRE WOODS, 1750 Tyson's Boulevard Suite 1800, McLean, VA, 22102-4215, US

(21) Appl. No.: 09/103,807

(22) Filed: Jun. 24, 1998

Related U.S. Application Data
(60) Provisional application No. 60/072,604, filed on Jan. 26, 1998.

(51) Int. Cl. A61K 48/00; C12N 5/06; A61K 39/00

(52) U.S. Cl. 424/93.1; 424/277.1; 435/325

(57) ABSTRACT

Homing bone marrow cells deposited at ATTC CRL-12424 can be transfected with a toxic gene which expresses a compound which alone, or in the company of a triggering agent, kills neighboring cancer cells in the bone marrow of a patient receiving the therapy. Toxic genes include cytotoxin such as thymidine kinase, immune stimulating compounds such as interleuken-2 and radiation repair inhibitors, such as Ku protein. The transfected cells can be administered directly to the site of the tumor or systemically, or regionally, intramedullary (into the marrow) through intravascular administration. The latter alternative permits the delivery of very high doses of the effective agent.

This application is a regular National application claiming priority from Provisional Application, U.S. application Ser. No. 60/072,604 filed Jan. 26, 1998. This application is related to U.S. patent application Ser. No. 08/785,008, allowed, U.S. patent application Ser. No. 08/990,746 (Attorney Docket 494-254-27, filed Dec. 15, 1997) and U.S. patent application Ser. No. 09/010,114 (Attorney Docket 494-280-27, filed Jan. 21, 1998). The disclosure of each of these U.S. Patent Applications is incorporated herein by reference.

### BACKGROUND OF THE INVENTION

1. Field of the Invention

This invention pertains to a therapeutic agent for the treatment of bone cancer, including osteosarcoma and metastatic bone cancer, particularly including prostate and breast cancer metastases. The invention also pertains to methods of administering the therapeutic agent effective in treatment of bone cancer. This agent is used alone, or together with an activating prodrug.

### BACKGROUND OF THE PRIOR ART

Prostate and certain breast cancer metastases to the skeleton as well as osteosarcoma cause significant mortality and morbidity for patients. While improved technology has facilitated both early diagnosis and prevention of this disease, over 50% of men with prostate cancer already have disseminated disease at the time of diagnosis. Detection of prostate cancer cells in bone marrow by reverse transcriptase-polymerase chain reaction (RT-PCR) of prostate-specific antigen (PSA) has been reported in men with pathologically organ-confined disease Wood et al. Cancer 74:2533, 1994.

Bone stromal cells can be molecularly engineered to express genes that will exert to bystander cell-kill upon the administration of a prodrug Cheon et al. Cancer Gene Therapy 4:359 (1997); Ko et al. Can Res. 56:1683-4614, 1996. Bone stromal cells can also be engineered to deliver a secretory paracrine growth factor fused with a toxin gene, which can be readily secreted to target prostate tumor cells that contain the appropriate receptor Miami et al. J. Urol 158:948, 1997. Further, bone stromal cells can be engineered to express factors that will activate cytotoxic NK cells (in athymic mice) and/or T lymphocytes (in syngeneic rats) in situ Vieweg et al. Cancer Res. 54:1760, 1994, which should elicit enhanced anti-tumor response in skeletal lesions. Bone marrow transfusion through intravenous injection is a common practice in the clinic to restore bone marrow function and to enhance drug resistance see Review. Maze et al. Mol. Med. Today 3:350, 1997.

### SUMMARY OF THE INVENTION

This invention, in its broadest aspects, encompasses a therapeutic agent effective in treating bone cancer, including originating bone cancer (osteosarcoma) as well as metastatic bone cancer, such as metastatic prostate and breast cancer. Other metastatic cancers that lodge in the skeleton may be treated with the therapeutic agent of this invention as well. The therapeutic agent relies on a bone marrow cell, originally isolated and cloned by inventors herein, and now deposited under accession number ATTC CRL-12424. This bone marrow cell is disclosed in detail in U.S. patent Ser. No. 08/990,746 (Attorney Docket 494-254-27, filed Dec. 15, 1997). The cell line was first described by Diduch et al., J. Bone and Joint Surgery 75:92-105 (1993). The description of the isolation and recovery of bone cells set forth on pages 93-94 thereof is superficial, and not sufficient to enable one of skill in the art to recover either the homing cell line, designated D1, or similar pluripotential cells. The whole disclosure of how to obtain these cells appears in the referenced application which has been incorporated herein. As disclosed in the referenced application of Balian et al., the D1 bone marrow cell may be effectively transfected with a variety of DNA sequences, effective in the expression of proteins. The previously-filed application is directed to, in part, transfected D1 cells effective in treating skeletal conditions other than cancer.

Certain of the inventors herein have previously demonstrated, U.S. application Ser. No. 08/785,008 allowed, and U.S. patent application Ser. No. 09/010,114 (Attorney Docket Number 494-280-27 CIP, filed Jan. 21, 1998) the effective treatment of tumors by administration of a recombinant adenovirus, transfected with a tissue/tumor restrictive promoter directing expression of a cytotoxic protein, such as thymidine kinase.

The Applicants herein have now discovered that these concepts may be combined, to effectively target particularly calcified cancers, including regional and metastatic cancers deposited in the bone. By transfecting the D1 cell line with a therapeutic gene under the control of an operable promoter, an “autohoming” therapeutic agent is provided, which can be administered to the cancer either locally by intramedullary injection (into the marrow), regionally through intravascular or local regional means, or systemically, such as through intravenous administration. Local-regional intravascular administration (through e.g., a catheter) provides for administration of higher local effective doses of adenoviruses.

The effective therapeutic gene is provided alone, or in conjunction with a companion agent, such as a prodrug, acyclovir or 5-Fluorocytosine (ACV and 5-FC respectively). These prodrugs are activated by thymidine kinase or cytosine deaminase thus exerting “bystander cell-kill”. In the case of paracrine regulation toxic genes and immunoregulators, the effective agent alone is expected to exert its individual biological effects. Because the cells home directly to the bone marrow, collateral damage is limited.

### DETAILED DESCRIPTION OF THE INVENTION

Specific aspects of the invention include the genetic engineering of a bone stromal cell line, D1, with thymidine kinase (TK) and cytosine deaminase (CD) genes; as well as a genetically engineered bone marrow stromal cell line, D1, with a secretory paracrine growth factor diphtheria toxin (DT) fusion protein under the positive regulation of a Tetracycline-inducible promoter (Tetp); genetically engineered bone stromal (D1) cell lines to express GM-CSF and IL12, IL-6 or IL2 in an effort to induce an immune response in skeletal tumor growth.

Preliminary Results:

Pluripotent D1 bone stromal cells were obtained from Balb-c mice bone marrow washes and were subjected to single cell cloning. These cells have the capability of differentiating into either osteoblasts, chondrocytes, or fat cells Diduch et al. J. Bone Joint Surg. 75:92, 1993, Cui et al. ibid 79:1054, 1997 U.S. patent application Ser. No. 08/990,746 (Attorney Docket Number 494-254-27, filed Dec. 15, 1997). D1 cells transfected with the β-galactosidase gene, when injected intravenously, were observed to home to the bone. D1 stromal cells were further genetically modified by transfecting these cells with a retroviral vector containing the TK gene. The resulting D1-TK cells, when co-cultured with either androgen-dependent or androgen-independent LNCaP and derivative cell lines, C4-2 and C4-2B, inhibited their growth in vitro in a co-cultured system. In a separate study, we observed that D1 cells have the capability of differentiating into mineralized bone when injected subcutaneously in athymic mice. D1-TK, when co-inoculated with C4-2 cells, formed chimeric tumors, and both the growth of these tumors and serum PSA were markedly depressed upon administration of ACV suggesting bystander cell-kill.

We have established that the osteocalcin (OC) promoter can mediate the expression of genes in a tumor-specific manner. OC-TK was found to be expressed by both prostate and bone tumor cells. Recombinant OC-TK adenovirus can direct the expression of TK and hence is cytolytic to osteosarcoma as well as its pulmonary deposits. This is disclosed in U.S. patent application Ser. No. 08/785,008, allowed, and U.S. patent application Ser. No. 09/010,114 (Attorney Docket Number 494-l 280-27 CIP, filed Jan. 21, 1998). Indeed, our laboratory has demonstrated that the adenovirus OC-TK can exert a tumoricidal effect when administered directly to skeletons that harbor either PC-3 or C4-2 tumors.

TK and CD, which are considered toxic genes, can cause cytotoxicity directly by converting the prodrugs ACV and 5-fluorocytosine (5-FC) into their active forms and inhibit, respectively, DNA synthesis in replicating cells and RNA synthesis in both replicating and non-replicating cells. Since prostate cancer cells are rapidly dividing when metastasizing to the skeleton, D1-TK or D1-CD exert bystander cell-kill of primarily prostate cancer cells and spare the damage to bone marrow cells. Specificity of genetically engineered bone cells to target prostate cancer bone metastasis relies on the fact that D1 cells have the unique ability to home to the skeleton in 2 to 3 weeks, at which time most of the D1 cells trapped in the liver and lung have been cleared. The paracrine growth factor DT fusion protein with IL13, the IL13-DT construct (kindly provided by Dr. R. Puri of the Food and Drug Administration), may be cloned into a plasmid containing Tetracycline (Tet)-inducible promoter. This version of the therapeutic gene binds to prostate cancer cells, which contain the IL13 receptor Maini et al. J. Urol. 158:948, 1997. The secretable form of IL13-DT, upon induction by Tetracycline, may bind to prostate cancer epithelium and cause cell death. IL13 is a glycosylated peptide with a molecular weight of 12,000, and it bears significant homology with the N- and C-termini of IL4 Minty et al. Nature 362:248, 1993. Receptors for hIL4 and hIL13 share a subunit that is responsible for intracellular signaling Obiri et al. JBC 270:8797, 1995. A wide range of human tumors express hIL4 and hIL13 receptors Debinski et al. JBC 268:14065, 193, Obiri et al. J. Clin. Invest. 91:88, 1993, Maini et al. J. Urol. 158:948, 1997. Pseudomonas exotoxin fusion protein with IL13 (closely related to DT-IL13 chimeric toxin) was shown to kill tumor cells, whether they were replicating or not, as long as they expressed receptors for hIL4 and hIL13. Paulus et al. J. Neurosurg. 87:89, 1997, demonstrated that the Tet system is 60-fold more responsive (to Tet) than the Lac (lactose) system in mediating DT expression in a human glioma tumor model. Transduced GM-CSF and IL12 plus IL2 infusion are effective cytokines in inducing local host anti-tumor immune response. Although athymic mice do not contain cytotoxic T cell activity, GM-CSF-IL12 in the presence of IL2 may induce NK cell activity, which potentially can be effective in eliciting local anti-tumor response.

Methods and Procedures:

General: Bone stromal cells are transfected with 3 types of therapeutic genes under the regulation of a universal cytomegalovirus (CMV) promoter or Tet-responsive element (TRE) driven by CMV promoter-mediated expression of a reverse Tc-responsive transcriptional activator (rtTA) (Tet-on system). D1 cells expressing TK have been shown to exert bystander cell-kill of prostate cancer cells. The tetracycline-inducible promoter can be activated in vivo and in vitro by the administration of tetracycline in drinking water to hosts after bone stromal cells have reached, propagated, and established in the bone microenvironment.

D1 cells are grown under conditions described in U.S. patent application Ser. No. 08/990,746 (Attorney Docket Number 494-254-27, filed Dec. 15, 1997). Cells are transfected with plasmid constructs containing either the CMV-TK or the -CD gene using DOTAP Zhau et al. CRC12:297, 1994, Marengo et al. Mol. Carcinog. 19:165, 1997. D1 cells are selected, cloned, expanded, and characterized with respect to their relative TK or CD activity. Because D1 cells do not form soft agar colonies, the bystander effect of D1 in affecting prostate epithelial cell growth can be assessed by anchorage-independent growth. D1-TK or -CD cells may be co-cultured with prostate epithelial cells in collagen gels. Bystander cell-kill may be determined by the addition of ACV or 5-FC to the cultured media. Alternatively, these results may be confirmed by co-culturing D1-TK or -CD cells with prostatic epithelial cells under two-dimensional growth conditions. The bystander effect of bone stromal cells in eradicating the growth of prostate cancer epithelial cells may be assessed by flow cytometry with LNCaP and C4-2 cells to be separated from bone stromal cells by a PSMA antibody.

This invention employs pluripotent bone stromal D1 cells as therapeutic gene carriers to target the growth of human prostate cancer cells in the skeleton. The basis of this approach is to take advantage of the homing characteristics of D1 cells, and to transduce this cell line stably with a gene encoding the expression of a protein effective in the treatment of bone cancer, operably linked to a promoter. A further example is an IL13-DT plasmid under the regulation of Tetracycline-inducible promoter (Clontech, Tet-on gene expression system). Two rounds of transfections are needed with pTet-on (G418 selection) plus pTRE-IL13-DT (hygromycin selection). The selected D1 cells are induced by Tetracycline to express and secrete IL13-DT. This soluble fusion protein is taken up by prostate cancer cells, which contain the IL13 receptor, through a receptor-mediated mechanism; DT contains domains which bind the DT receptor (this domain has been replaced by IL13), translocate toxin into the cytosol, and inhibit protein synthesis and cell growth through the blockade of ADP ribosylation of elongation factor-2 Pastan et al. Biochem. 651:331, 1992. It has been shown that both androgen-dependent and -independent prostate cancer cells contain high affinity cell-surface IL13 receptors Maini et al. J Urol 158:948, 1997, whereas most of the normal tissues, with few exceptions (e.g. monocyte and B cells) Zurawski et al. Immunol. Today 15:19, 1994, do not express the IL13 receptor. Thus, this fusion protein upon induction by Tet may exert bystander cell-kill against the neighboring prostatic cancer epithelial cells which reside in the bone marrow; D1 cells do not contain the IL13 receptor, and thus serve as an efficient gene carrier cell line.

Toxic gene therapy with such agents as TK, DT, or CD induces tumor regression through direct induction of apoptosis. Immune effector cells can also elicit tumor destruction, through both inflammatory and cytotoxic mechanisms. Cellular mediators of anti-tumor activity include cytotoxic T lymphocytes (CTLs) and Natural Killer (NK) cells, both of which have demonstrated anti-tumor activity (Nabel Brit. J. Surg. 79:990, 1992, Talmadge Biother 4:215, 1992. Whereas CTLs depend upon HC class I activation and presentation of tumor antigens by antigen presenting cells (APCs), NK cells do not. GM-CSF and IL2 have been shown to activate host anti-prostate immune response in syngeneic animals Vieweg et al. Cancer Res. 54:1760, 1994, Sanda et al. J. Urol. 151:622, 1994, Moody et al. Prostate 24:244, 1994. IFN-α and Interleukins (IL)2, 4 and 12 enhance both NK and CTL activity Redmond et al. J. Surg. Res. 52:406, 1992, van Moorselaar et al. Prostate 8:331, 1991. APC proliferation can also be induced by combinations of IL4, GM-CSF, and IFN-α (Romani J. Expt. Med. 180:83, 1994. Whereas IL4 and 12 have local anti-tumor effects, IL2 and GM-CSF augment systemic immunity (Tepper Bone Marrow Transp. 9(Supp.):177, 1992, Dranoff et al. PNAS 90:3539, 1993.

The D1 bone marrow cell line that acts as the gene transport and delivery agent in this invention can be accessed through accession number CRL-12424. The deposit was first made Oct. 28, 1997. The deposit at the American Type Culture Collection, 10801 University Blvd., Manassas, Va. 22110, was made pursuant to the Budapest Treaty, and all restrictions thereon will be removed upon issuance of a patent on this application, if not previously removed.

D1 cells home to the bone marrow of a mammal, and once located in the bone marrow, express DNA within the cell. Methods of preparing bone marrow cell lines of this type are set forth in co-pending U.S. patent application Ser. No. 08/990,746. (Attorney Docket Number 494-254-27). Methods of transfecting this cell line are set forth in the referenced application, the totality of which has been incorporated herein by reference.

The preparation of recombinant virus (adenovirus) together with an operational promoter and a cytotoxic agent which converts the prodrug ACV, resulting in TK-induced killing of adjacent tumor cells, is disclosed in allowed U.S. patent application Ser. No. 08/785,088 and U.S. patent application Ser. No. 09/010,114, (Attorney Docket Number 494-280-27 CIP), as well as demonstrating the effectiveness of administration of agents of this type either directly, systemically through intravascular administration, such as intravenous administration, as well as regionally or intramedullary (through a hole in the bone cortex) or through the intercondylar notch or through intravascular administration using a catheter. The latter alternative permits for increased local dosages.

The same promoter/protein combinations may be used to transfect D1 bone marrow cells. Suitable promoters include, in addition to the osteocalcin promoter, a tetracycline-inducable promoter (Clontech, Tet-on gene expression system), a cytomegalovirus (CMV) promoter or other commercially available promoter.

The DNA selected to encode an anti-tumor agent can be selected from a wide variety of agents. As noted above, TK and CD can be administered with ACV and 5-FC to achieve significant tumor reduction. A secretory paracrine growth factor/diphtheria toxin (DT) fusion protein is also effective. Advantagously, this is placed under the positive regulation of a tetracycline-induceable promoter (Tetp). Alternative agents to be expressed include granulocyte-macrophage colony-stimulating factor (GM-CSF) or other immune system stimulating proteins, including interleukin-2, interleukin-4, interleukin-12 and interleukin-6.

Additional effective agents include kanamycin kinase (KK). Further, the DNA selected to transfect the D1 cell may be selected so as to encode the expression of a factor which impairs repair mechanisms following radiation-induced DNA damage in cells. Specifically, compounds have been identified which bind to DNA fragments severed under the impact of cancer radiation therapy. The fragments exhibit bound severed ends and can not be repaired or recombined by the machinery of the tumor cell. One such recently cloned enzyme, Ku protein, is described in Cancer Research 57:1412-1415 (1997). This is another effective agent, and the article is incorporated herein by reference.

The above examples are representative in nature, and not exhaustive. Similarly, dosage levels will vary widely depending on the patient, the active agent selected for expression, the promoter, etc. As a general rule, effective tumor reduction has been established at levels of about 1×10⁸ up to 5×10⁹ plaque-forming units (PFU) per 40-75 microliters, coupled with ACV administration of from 20-80 mg/kg body weight per day, preferably 30-50 mg/kg per day. Other levels can be adjusted from this starting point.

This invention embraces the reduction in tumor size and activity, as well as the elimination of bone cancer.

Having disclosed the invention both generically and by example, the invention should not be limited by the examples except for restrictions in the claims expressly set forth, below. In particular, other modes of administration, dosage levels and the like will be obtained by those of ordinary skill in the art without the exercise of inventive skill. Such alternatives remain within the scope of the invention, as set forth in the claims below.

What is claimed is:

1. A therapeutic agent, comprising a bone marrow cell which, when introduced into a mammal, homes to bone marrow of said mammal, wherein said bone marrow cell has been transfected with DNA operably connected to a promoter, which DNA encodes the expression of an anti-tumor agent selected from the group consisting of cytotoxic agents, an immune response stimulating agent and a radiation sensitive repair inhibitor.

2. A therapeutic agent, comprising a bone marrow cell which, when introduced into a mammal, homes to bone marrow of said mammal, wherein said bone marrow cell has been transfected with DNA operably connected to a promoter, which DNA encodes the expression of an agent selected from the group consisting of thymidine kinase (TK), cytosine deaminase (CD), a secretory paracrine growth factor/diphtheria toxin fusion protein (DT), granulocyte-macrophage colony-stimulating factor (GM-CFS), interleukin-2, interleukin-6, interleukin-12, kanamycin kinase, and Ku protein.

3. The therapeutic agent of claims 1 or 2, wherein said bone marrow cell has the bone marrow homing characteristics of a cell line deposited under accession number ATTC CRL-12424.

4. The therapeutic agent of claim 3, wherein said bone marrow cell is obtained, either directly or through sub-cloning, from the cell line deposited under accession number ATTC CRL-12424.

5. A method of treating bone cancer in a mammal, comprising administering to said mammal a therapeutically effective amount of the therapeutic agent of claim 1.

6. The method of claim 5, wherein said administration is directly to said bone cancer.

7. The method of claim 5, wherein said administration is to said mammal, systemically.

8. The method of claim 5, wherein said administration is provided to a region of tissue affected by a tumor or it metastasis.

9. The method of claim 5, wherein said bone cancer is osteosarcoma, metastatic prostate cancer or metastatic breast cancer.
